(19) United States
(12) Patent Application Publication
(10) Pub. No.: US 2011/0185611 A1
(43) Pub. Date: Aug. 4, 2011

(54) APPARATUS AND METHOD FOR RAPID IDENTIFICATION AND TRACKING OF INDIVIDUALS IN AN EMERGENCY SITUATION

(75) Inventors: Nicky Bruce Adams, Evans, GA (US); Adam Neil Buckley, Marietta, GA (US); Mark Dennis Goodell, Martinez, GA (US); Dewey Thomas Holland, Martinez, GA (US); Stanley J. Kuzia, Evans, GA (US)

(73) Assignee: GLOBAL EMERGENCY RESOURCES, LLC, Augusta, GA (US)

(21) Appl. No.: 12/696,287

(22) Filed: Jan. 29, 2010

(51) Int. Cl. A44C 5/00; A61B 5/117; H04Q 5/22; G06K 19/06

(52) U.S. Cl. 40/633; 340/10.1; 235/494

(57) ABSTRACT

An apparatus and method for rapid identification and tracking of individuals in an emergency situation is disclosed. The apparatus may comprise a spring loaded band that includes a spring element movable between a first stable position and a second stable position. The spring element may be configured to coil lengthwise around an individual's wrist, arm, ankle or leg while transitioning from the second stable position to the first stable position. A hygienic coating may be configured to cover the spring element. Finally, a unique patient identifier number may be disposed on the spring loaded band.

### FIELD OF THE INVENTION

The present subject matter relates generally to an apparatus and method for rapid identification and tracking of individuals in an emergency situation and particularly to a spring loaded band for use in patient triage, hospital tracking, and mass evacuations that may be quickly attached and secured to an individual.

### BACKGROUND OF THE INVENTION

In emergency situations, such as an injury causing accident, a terrorist attack, or a mass evacuation, it is necessary to be able to quickly identify the non-injured, injured, evacuees, incident responders, and the like. In particular, the medical condition of the injured often needs to be quickly identified to enable first responders to efficiently and effectively provide medical care. Moreover, properly identifying the persons involved in an emergency situation is necessary to permit such persons to be accurately tracked as they are transferred from the scene of the emergency to a hospital, an evacuation point, or some other similar location.

Typically, first responders to the scene of an accident use a triage tag, secured to an accident victim's wrist, to quickly note the injury type, vital signs and severity of the injuries of a victim. Triage tags generally consist of paper or vinyl cards secured by string or tether or plastic bands secured with snaps, slots, or similar fastening mechanisms. For example, conventional triage tags require emergency responders to loop a strap around a victim's wrist, pull the strap through a slot, remove a covering off of the end of the strap to expose an adhesive section, and then affix the adhesive section to a portion of the tag to fully secure the triage tag to the victim. Such two-handed operation is typically required for most conventional triage tags, which generally wastes precious time at the scene of the accident and can frustrate the efforts of the emergency responders. Moreover, conventional triage tags are susceptible to falling off or being damaged during transport of a victim to a nearby hospital or emergency care facility.

Accordingly, there is a need for a band that may be quickly attached and secured to an individual to enable such individual to be properly identified and tracked during and/or following an emergency situation.

### BRIEF DESCRIPTION OF THE INVENTION

Aspects and advantages of the present subject matter will be set forth in part in the following description, or may be obvious from the description, or may be learned through practice of the present subject matter.

In general, the present subject matter is directed to an apparatus and method for rapid identification and tracking of individuals in an emergency situation. For example, the present subject matter discloses a spring loaded band for use in patient triage and hospital tracking that may be quickly attached and secured to an individual. The band is also suitable for use in various other applications. For example, the band may be suitable for use during and/or following a mass evacuation or a terrorist attack.

In one embodiment of the present subject matter, a spring loaded band can include a spring element movable between a first stable position and a second stable position, the spring element being configured to coil lengthwise around an individual's wrist, arm, ankle or leg while transitioning from the second stable position to the first stable position; a hygienic coating being configured to cover the spring element; and a unique patient identifier number disposed on the band.

In this aspect of the present subject matter, the spring element may comprise a metal bi-stable ribbon spring.

Also in this aspect of the present subject matter, the hygienic coating may comprise a hygienic plastic or vinyl coating.

Further in this aspect of the present subject matter, the patient identifier number may be printed, molded or etched onto the hygienic coating.

In this aspect of the present subject matter, the spring loaded band may include a barcode disposed on the hygienic coating. The barcode may correspond to the patient identifier number.

Also in this aspect of the present subject matter, the color of the hygienic coating may be varied so as to correspond to an individual's condition or triage status.

Further in this aspect of the present subject matter, the spring loaded band may include an RFID tag configured to transmit a signal corresponding to the patient identifier number. The RFID tag may be disposed on the band or embedded within the band.

In this aspect of the present subject matter, the spring loaded band may include a removable tag secured to the hygienic coating. The patient identifier number may be disposed on the removable tag.

Also in this aspect of the present subject matter, the removable tag may include a barcode corresponding to the patient identifier number.

Further in this aspect of the present subject matter, the removable tag may include a RFID tag configured to transmit a signal corresponding to the patient identifier number.

In this aspect of the present subject matter, the spring loaded band may include a color coded label removably secured to the hygienic coating. The color of the color coded label may be varied so as to correspond to an individual's condition or triage status.

Also in this aspect of the present subject matter, the spring loaded band may include a detachable extension configured so as to be removable in stages to indicate or update an individual's condition or triage status.

Further in this aspect of the present subject matter, the spring loaded band may include a biometric sensor configured to sense and transmit health readings of an individual.

In this aspect of the present subject matter, the spring loaded band may include a fastening mechanism disposed on the band, wherein the fastening mechanism comprises a VELCRO strip and a corresponding mating surface or a strip of adhesive.

In another embodiment of the present subject matter, a method for identifying and tracking individuals during patient triage, at a hospital or during a mass evacuation is provided. The method can include affixing a spring loaded band to an individual by applying a force to the band so as to initiate a self-transition of the band from a second stable position to a first stable position, wherein the band is configured to coil lengthwise around the individual's wrist, arm, ankle or leg while transitioning to the first stable position; and assigning a patient identifier number associated with the band to the individual so as to permit such individual to be identified and tracked.

In this aspect of the present subject matter, the method may include selecting the spring loaded band to be affixed to the individual so that the color of the band corresponds to the individual's condition or triage status, wherein the color of the band may be varied to correspond to a particular condition or triage status.

Also in this aspect of the present subject matter, the method may include securing a color coded label to the spring loaded band so as to indicate or update the individual's condition or triage status, wherein the color of the color coded label may be varied to correspond to a particular condition or triage status.

Further in this aspect of the present subject matter, the method may include securing a removable tag to the spring loaded band, wherein the patient identifier number is disposed on the removable tag.

In this aspect of the present subject matter, the removable tag may include a barcode corresponding to the patient identifier number and a RFID tag configured to transmit a signal corresponding to the patient identifier number.

Also in this aspect of the present subject matter, the method may include removing tabs from a detachable extension of the spring loaded band so as indicate or update the condition or triage status of the individual.

Further in this aspect of the present subject matter, the method may include sensing an individual's vital health information with a biometric sensor disposed on the spring loaded band.

In this aspect of the present subject matter, the method may include securing a fastening mechanism to the band.

These and other features, aspects and advantages of the present subject matter will become better understood with reference to the following description and appended claims. The accompanying drawings, which are incorporated in and constitute a part of this specification, illustrate embodiments of the present subject matter and, together with the description, serve to explain the principles of the present subject matter.

### BRIEF DESCRIPTION OF THE DRAWING

A full and enabling disclosure of the present subject matter, including the best mode thereof, directed to one of ordinary skill in the art, is set forth in the specification, which makes reference to the appended figures, in which:

FIG. 1 illustrates an exploded front view of an embodiment of a spring loaded band in accordance with an aspect of the present subject matter;

FIG. 2 illustrates an exploded side view of an embodiment of a spring loaded band in accordance with an aspect of the present subject matter;

FIG. 3 illustrates an perspective view an embodiment of a spring element of the spring loaded band in a first stable position in accordance with an aspect of the present subject matter;

FIG. 4 illustrates an perspective view of an embodiment of a spring element of the spring loaded band in a second stable position in accordance with an aspect of the present subject matter;

FIG. 5 illustrates a top view of an embodiment of a spring loaded band with a removable tag in accordance with an aspect of the present subject matter;

FIG. 6 illustrates a top view of an embodiment of a spring loaded band with a color coded label in accordance with an aspect of the present subject matter;

FIG. 7 illustrates a top view of an embodiment of a spring loaded band with a detachable extension in accordance with an aspect of the present subject matter;

FIG. 8 illustrates a simplified, partial cross-sectional view of an embodiment of a spring loaded band with a biometric sensor in accordance with an aspect of the present subject matter, particularly illustrating the band coiled around an individual's wrist;

FIG. 9 illustrates a side view of an embodiment of a spring loaded band with a fastening mechanism in accordance with an aspect of the present subject matter; and

FIG. 10 illustrates a side view of another embodiment of a spring loaded band with a fastening mechanism in accordance with an aspect of the present subject matter.

### DETAILED DESCRIPTION OF THE INVENTION

Reference now will be made in detail to embodiments of the present subject matter, one or more examples of which are illustrated in the drawings. Each example is provided by way of explanation of the present subject matter, not by way of limitation. In fact, it will be apparent to those skilled in the art that various modifications and variations can be made in the present subject matter without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

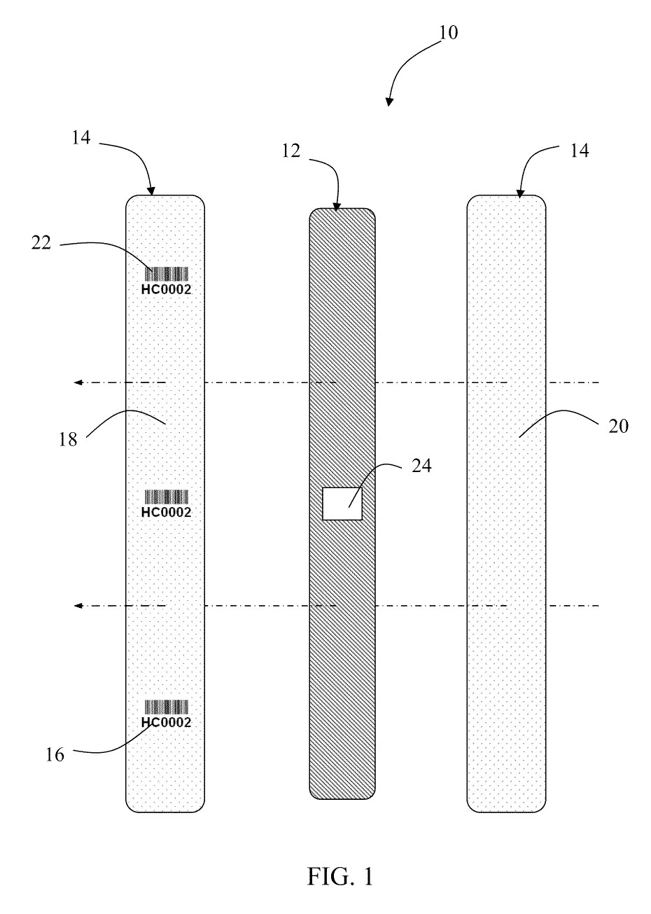

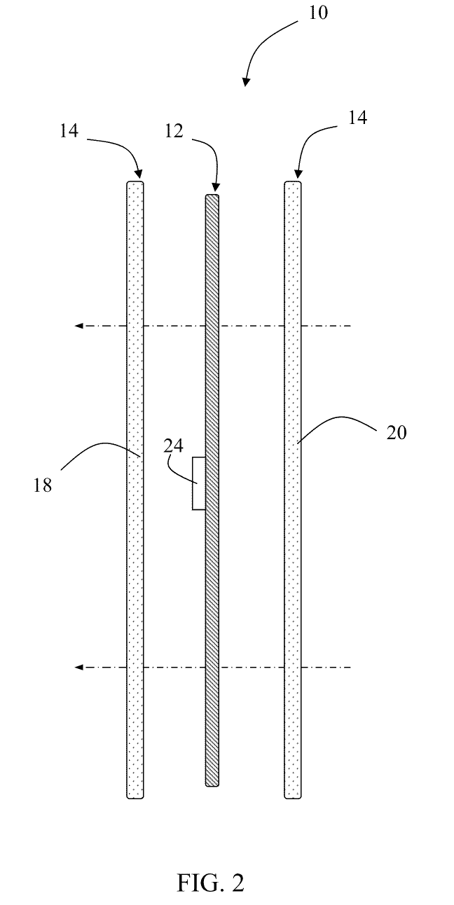

In accordance with an aspect of the present subject matter, FIGS. 1 and 2 illustrate exploded front and side views of an embodiment of a spring loaded band 10 for use in patient triage and hospital tracking, respectively. Initially, it should be appreciated that, although the spring loaded band 10 may be generally described herein as being used in patient triage and hospital tracking, the band 10 may also be utilized in various other settings to provide a means for identifying and tracking persons, such as after a terrorist attack or during a mass evacuation.

In general, the spring loaded band 10 may be configured so as to be quickly attached and secured to an individual's arm, wrist, leg, ankle or other body part. Thus, the band 10 may include a spring element 12 movable between first and second stable positions. Additionally, the spring element 12 may be configured to coil lengthwise around an individual's wrist, arm, ankle or leg while transitioning to the first stable position. The spring loaded band 10 may also include a hygienic coating 14 configured to cover the spring element 12. Further, a unique patient identifier number 16 may be disposed on the band 10.

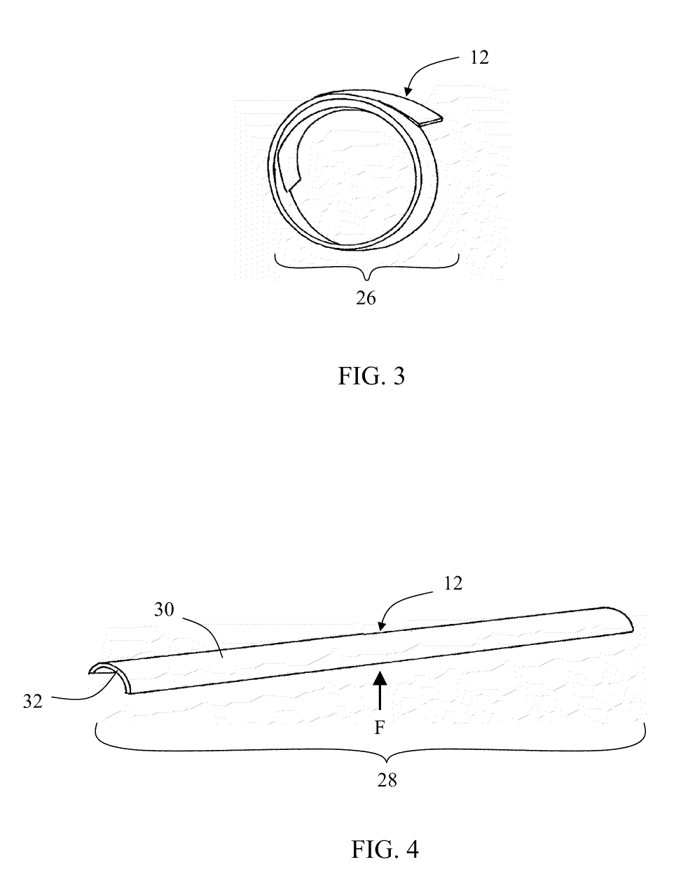

FIGS. 3 and 4 illustrate an embodiment of a spring element 12 in accordance with an aspect of the present subject matter. The spring element 12 may be configured to be movable between a first stable position 26 (FIG. 3) and a second stable position 28 (FIG. 4). For example, the spring element 12 may generally comprise a bi-stable, spring loaded strip of metal or plastic, such as a bi-stable ribbon spring, configured to be coiled along its longitudinal axis in the first stable position 26 and flattened with an arcuate cross-section in the second stable position 28. Thus, as seen in FIG. 3, the spring element 12 may be normally in a first “coiled” stable position 26. In particular, the spring element 12 may be designed to have a tendency, due to longitudinal stresses within the spring element 12, to normally coil lengthwise upon itself so as to form a circular band. However, as shown in FIG. 4, the spring element 12 may also be uncoiled to permit the spring element 12 to assume the second “linear” stable position 28. In this position, the spring element 12 may generally have an arcuate cross-section, including a concave side 30 and a convex side 32, which holds the spring element 12 straight and resists the spring element's 12 tendency to coil upon itself. Specifically, the arcuate cross-section may result in a bending moment of inertia that resists the longitudinal coiling stresses created by uncoiling the spring element 12 from the first stable position 26. As such, the spring element 12 may be maintained in the second stable position 28 despite the tendency to coil upon itself.

From the second stable position 28, the spring element 12 may be quickly and easily returned to the first stable position 26 by the application of a bending or buckling force F (FIG. 4) to the convex side 32 of the spring element 12. For example, simply pressing, pushing, or slapping the convex side 32 of the spring element 12 against the wrist, arm, ankle or leg of an individual may be generally sufficient to initiate self-coiling of the spring element 12. The spring element 12 may then continue to self-transition to the first stable position 26, either fully coiling lengthwise upon itself or, when used in accordance with an aspect of the present subject matter, coiling lengthwise around an individual's wrist, arm, ankle or leg. This self-coiling action, thus, makes application of the spring element 12 in patient triage particularly desirable, since emergency responders must be able to quickly tag the injured and non-injured. Moreover, as the spring element 12 may be configured to coil upon itself, the spring loaded band 10 may be automatically secured to an individual without the necessity of a fastening mechanism. Thus, the self-latching action of the spring element 12 can ensure that the spring loaded band 10 remains properly secured to an individual during transport of such individual to a hospital or other health care facility.

Referring back to FIGS. 1 and 2, the spring element 12 may be covered by a hygienic coating 14. The hygienic coating 14 may include a top layer 18 and a bottom layer 20 configured to completely cover the spring element 12 when the spring loaded band 10 is assembled. Thus, it should be appreciated that the top and bottom layers 18, 20 may have dimensions that are slightly larger than those of the spring element 12 so as to permit the hygienic coating 14 to fully cover the spring element 12. It should also be appreciated that the layers 18, 20 may be attached to the spring element 12 and/or to one another by any suitable means. For example, in one embodiment, the top and bottom layers 18, 20 may be secured to one another around the perimeter of the spring element 12 by heat sealing the hygienic coating material or by the application of adhesive. In an alternative embodiment, the hygienic coating 14 may comprise a single component, rather than separable layers. For example, the hygienic coating 14 may be formed by molding the coating material around the spring element 12 by methods generally known in the art.

In a preferred embodiment, the hygienic coating 14 may be composed of a plastic or vinyl hygienic material. However, it should be appreciated that the hygienic coating 14 may be generally composed of any material that can be used to prevent mold, bacteria, or other harmful micro-organisms from collecting or growing on the spring loaded band 10. Such a hygienic coating 14 may be particularly desirable in a triage application, as the outside of the band 10 may be exposed to the open wounds of injured patients. Additionally, the hygienic coating 14 may be used to prevent direct exposure of an individual's skin to the spring element 12. For example, the coating 14 can minimize the likelihood of injury due to the spring element 12 rubbing against an individual's wrist, arm, leg, ankle or other body part.

Further, as indicated above, a unique patient identifier number 16 may be disposed on the spring loaded band 10. This may be included so that emergency responders and medical service providers can properly identify an individual while still complying with federal laws regulating the privacy of certain health information. It should be appreciated that the patient identifier number 16 may be generally disposed at any location on the band 10. In one embodiment, illustrated in FIG. 1, the patient identifier number 16 may be disposed at various locations on the top layer 18 of the hygienic coating 14. Further, it should also be appreciated that the patient identifier number 16 may be formed on the band 10 by any suitable means. For example, the patient identifier number 16 may be molded, printed or etched onto the hygienic coating 14.

Referring still to FIG. 1, the spring loaded band 10 of the present subject matter may also include one or more barcodes 22. In general, each barcode 22 may correspond to the unique patient identifier number 16 and may be configured to be read by a barcode scanner or any other suitable optical scanning device known in the art. As shown, the barcodes 22 may be disposed on a top layer 18 of the hygienic coating 14, such as by molding or printing the barcode 22 on the coating 14. However, it should be appreciated that the barcodes 22 need not be disposed on the top layer 18 as illustrated in FIG. 1, but may generally be disposed at any location on the band 10 at which a barcode 22 may be scanned. Additionally, one of ordinary skill in the art should appreciate that, generally, any type of barcode 22 may be utilized within the scope of the present subject matter. For instance, to provide several non-limiting examples, the barcode 22 may comprise a numeric-only barcode, an alpha-numeric barcode or a 2-dimensional barcode.

In addition to the inclusion of a barcode 22 or as an alternative thereto, the spring loaded band 10 may also include a Radio-Frequency Identification (RFID) tag 24 disposed on the band 10 or embedded within the band 10. A RFID tag 24 may be configured to transmit a radio-frequency signal. Thus, in accordance with an aspect of the present subject matter, the RFID tag 24 may be configured to transmit a signal corresponding to the unique patient identifier number 16 associated with a particular band 10. Such a configuration may allow emergency responders to identify and/or track multiple persons simultaneously during an emergency situation.

It should be appreciated that the RFID tag 24 may be generally disposed at any location on the spring loaded band 10. As illustrated in FIGS. 1 and 2, the RFID tag 24 may be secured to the spring element 12 so as to be embedded in the band 10 between the spring element 12 and the hygienic coating 14. Additionally, one of ordinary skill in the art should appreciate that the RFID tag 24 may be secured or attached to the band 10 by any suitable means. For example, in one embodiment, the RFID tag 24 may be secured to the spring element 12 by adhesive.

The use of barcodes 22 and/or RFID tags 24 in the spring loaded band 10 of the present subject matter may be useful in providing quick and easy tracking of injured patients or, in the case of a mass evacuation, evacuees. For example, first responders to an emergency situation may be equipped with handheld devices capable of scanning the barcodes 22 and/or receiving transmissions from the RFID tags 24 disposed on or embedded within an individual's band 10. Preferably, the handheld device may be configured to run a software tracking package, which can allow medical providers to quickly input patient information corresponding to the scanned or received patient identifier number 16 and share such information with the hospital, health care system, shelter, or similar location to which the injured patient or evacuee is being transported. As an example, one suitable, commercially available software package includes PATIENT TRACKING SOLUTION® by GLOBAL EMERGENCY RESOURCES™, LLC (Augusta, Ga.).

In yet a further embodiment of the present subject matter, the particular color of a spring loaded band 10 may be varied so as to correspond to the condition or triage status of an individual. For instance, the hygienic coating 14 may be provided in a plurality of colors, with each color signifying a condition or status in a chosen color code. Thus, as a non-limiting example, the hygienic coating 14 of a particular band 10 may be one of the following colors so as to correspond to the following patient condition or triage status: green to indicate that the individual should be held at a particular location, yellow to indicate that the individual is ready for transport or should be transported, red to indicate that the individual's status is critical, black to indicate that an individual is deceased or white to indicate that an individual is non-injured. Of course, it should be appreciated that that the spring loaded band 10 may be provided in numerous other colors so as to correspond to various other patient and/or triage conditions.

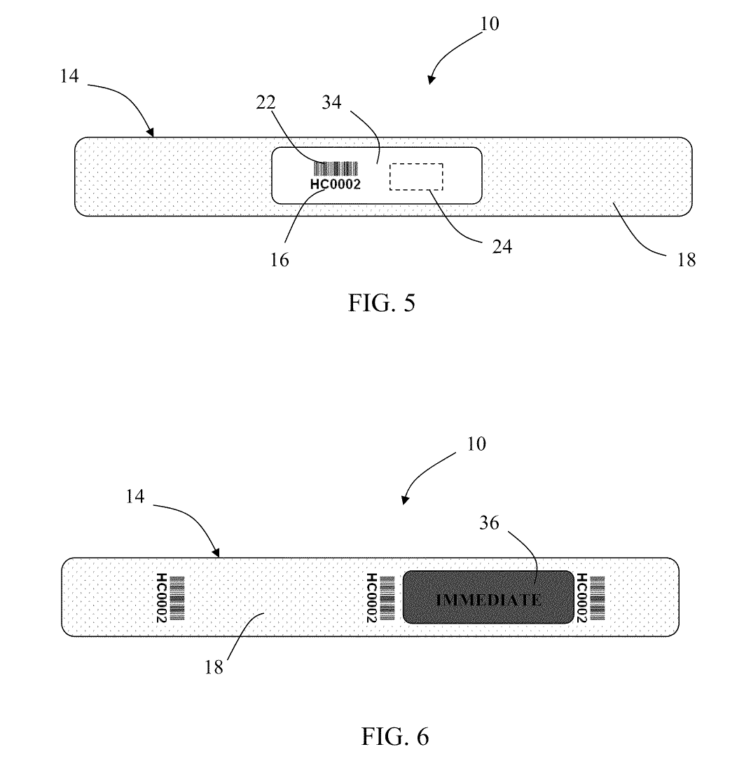

In still another embodiment of the present subject matter, illustrated in FIG. 5, the spring loaded band 10 may also include a removable tag 34. In general, the tag 34 may be removably secured to the band 10 by any suitable means. For instance, the tag may be removably secured to the hygienic coating 14 via adhesive, tether or VELCRO. Additionally, as is shown in the illustrated embodiment, the patient identifier number 16 may be disposed on the removable tag 34. Thus, it should be appreciated that the patient identifier number 16 may be disposed on the removable tag 34 in addition to, or as an alternative to, locating the patient identifier number 16 on the hygienic coating 14 (FIG. 1).

Moreover, still referring to FIG. 5, the removable tag 34 may also include a barcode 22 and/or a RFID tag 24, both of which may correspond to the patient identifier number 16 as generally described above. In particular, the barcode 22 may be disposed on a front surface of the removable tag 34 so as to be capable of being scanned by an optical scanning device. Additionally, the RFID tag 24 may be disposed on a front or back surface of the removable tag 34 or embedded within removable tag 34, itself.

In a further embodiment of the present subject matter, a color coded label 36 may be removably secured to the spring loaded band 10. For example, as shown in FIG. 6, the color coded label 36 may be attached to the top layer 18 of the hygienic coating 14. It should be appreciated that the color coded label 36 may be removably secured to the band 10 by any means generally known in the art. In one embodiment, the color coded label 36 may be attached to the hygienic coating 14 via adhesive, tether, or VELCRO.

The particular color of a color coded label 36 may be generally varied so as to correspond to a certain condition or triage status. In a preferred embodiment, the color code used for the color coded label 36 may be same as the color code used for the spring loaded band 10. Thus, referring to the embodiment described above, the color coded label 36 may be one of the following colors so as to signify the following condition or triage status: green to indicate that the individual should be held at a particular location, yellow to indicate that the individual is ready for transport, red to indicate that the individual's status is critical, black to indicate that an individual is deceased and white to indicate that an individual is non-injured. In addition, the color coded label 36 may also include the corresponding condition or triage status printed directly on the label 36. Thus, as seen in FIG. 6, the color coded label 36 may include language such as “IMMEDIATE” so as to clearly indicate the condition or status of a patient to an emergency responder who may not be familiar with the chosen color code.

One of ordinary skill in the art should appreciate that, since the color coded label 36 may be configured to be easily attached to and removed from the band 10, the label 36 may also be used to indicate a change in the condition or triage status of an individual so to provide flexibility in monitoring persons at the scene of an accident. For example, a patient at the scene of an accident may be initially examined and a white spring loaded band 10 may be secured to the patient to signify that the patient is not injured. However, it may become apparent after a short period of time that the patient has actually suffered an injury. At that point, an emergency responder or medical service provider may then secure a particular color coded label 36 to the band 10 in order to properly indicate and update the patient's condition.

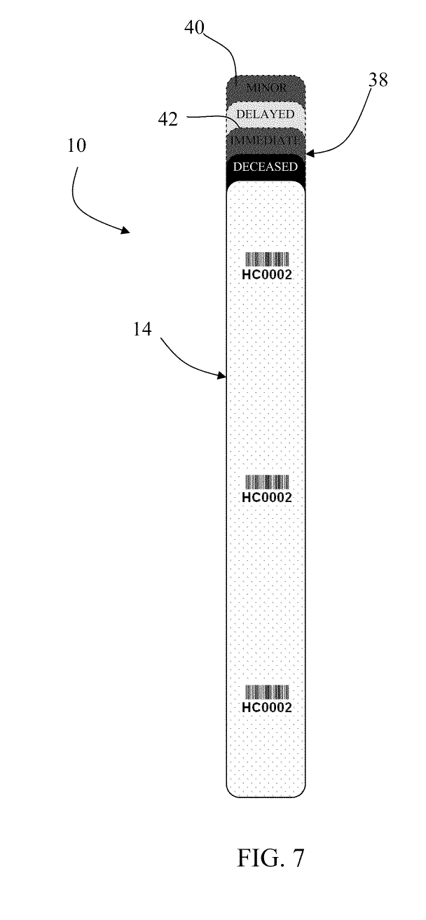

In yet still another embodiment of the present subject matter, shown in FIG. 7, the spring loaded band 10 may also include a detachable extension 38 configured so as to be removable in stages to indicate or update an individual's condition or triage status. One of ordinary skill should appreciate that the detachable extension 38 may be formed as an integral part of the band 10 or may be secured to the band 10 as a separate component. For example, in the illustrated embodiment, the detachable extension 38 may form an integral extension of the hygienic coating 14. Additionally, to permit the detachable extension 38 to be removable in stages, the extension 38 may be segmented into a plurality of separable tabs 40. It should be appreciated that the tabs 40 may be color coded so as to correspond to a particular triage status or patient condition. For example, in one embodiment, the same color code as that described above for the color coded label 36 and the hygienic coating 14 may be used for the tabs 40. Further, the tabs 40 may include a particular condition or triage status printed on the face of each tab 40 so as to clearly indicate the condition or triage status to an emergency responder.

Moreover, the detachable extension 38 may be configured such that each of the tabs 40 may be separately removed from the band 10. For example, as illustrated in FIG. 7, the detachable extension 38 may include perforated edges 42, represented by the dashed lines, to allow the tabs 40 to be separately detached from the band 10, and thus, permit an emergency responder to quickly indicate or update an individual's condition or triage status. It should be appreciated, however, that the tabs 40 may be generally configured to be detached from the spring loaded band 10 by any means and, thus, the detachable extension 38 need not include perforated edges 42.

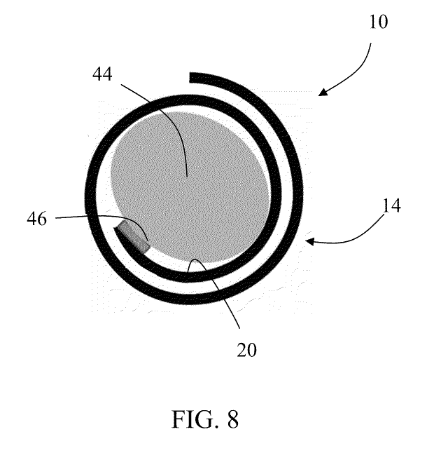

Referring now to FIG. 8, a further embodiment of the present subject matter is illustrated. As shown, the spring loaded band 10 may also include a biometric sensor 46. In general, the biometric sensor 46 may be configured to sense and transmit vital health readings of an individual wearing a band 10. For example, the biometric sensor 46 may be configured to sense an individual's heartbeat and/or temperature and then transmit this health information to a device configured to receive and process such information. For instance, the biometric sensor 46 may include an antenna, similar to that of an RFID tag, to permit the sensor 46 to transmit health information wirelessly. Such a configuration can provide a means for allowing emergency responders to continuously monitor vital health information of one or more individuals during and/or following an emergency situation.

To ensure that the health readings taken by the biometric sensor 46 are accurate, it may be desirable for the sensor 46 to be disposed on the spring loaded band 10 so as be in direct contact with an individual's wrist, arm, ankle or leg. Thus, in the embodiment shown in FIG. 8, the biometric sensor 46 may be attached to the outside of the bottom layer 20 of the hygienic coating 14 so as to come in direct contact with a patient's wrist 44. Moreover, due to the self-coiling nature of the spring element 12, the band 10 may exert a constant pressure on the location at which the band 10 is secured to an individual. Thus, in the illustrated embodiment, this constant pressure can maintain the biometric sensor 46 in contact with an individual's wrist 44 so as to allow the sensor 46 to continuously monitor the individual's vital health information.

It should be appreciated that, although the spring loaded band 10 may be configured to be self-latching, the band 10 may also include an fastening mechanism so as to further secure the band 10 to an individual. For example, in one embodiment, the band 10 may include a fastening mechanism in order to maintain the pressure exerted by the band 10 on an individual's wrist or other body part at a certain level. This may be desirable, for instance, to allow a biometric reader 46 disposed on the band 10 to continuously monitor an individual's vital health information. Additionally, a fastening mechanism may be included to reduce the risk of the spring loaded band 10 being tampered with or accidentally removed.

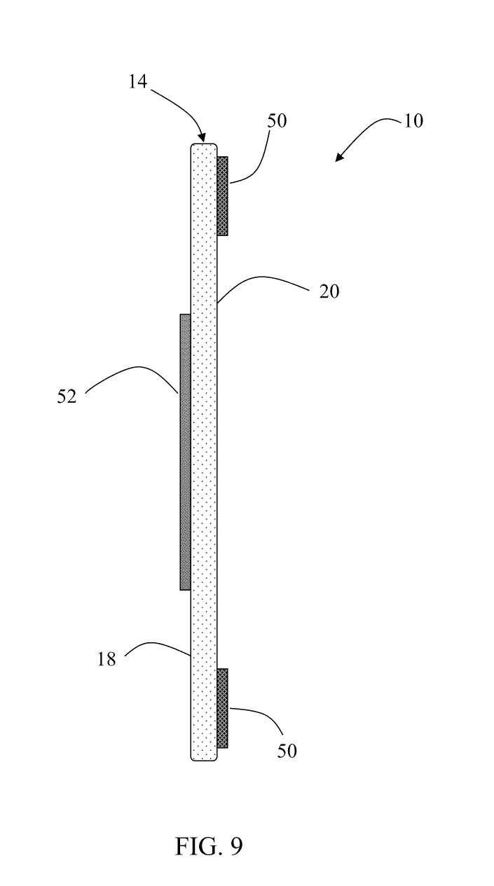

One of ordinary skill in the art should appreciate that the fastening mechanism may take various forms and may comprise any suitable attachment or fastening means generally known. In one embodiment, the fastening mechanism may comprise at least one strip of VELCRO 50 and a corresponding mating surface 52. In general, the VELCRO strip(s) 50 may be disposed at any location on the spring loaded band 10 such that the VELCRO 50 mates and locks together with the mating surface 52 when the band 10 is coiled upon itself For example, as shown in FIG. 9, a pair of VELCRO strips 50 may be disposed at opposing ends of the bottom layer 20 of the hygienic coating 14 and a corresponding mating surface 52 may be disposed on the top layer 18 of the hygienic coating 20. As such, one of the VELCRO strips 50 can mate with and be secured to the mating surface 52 when the band 10 is coiled around an individual's wrist, arm, ankle, leg or other body part.

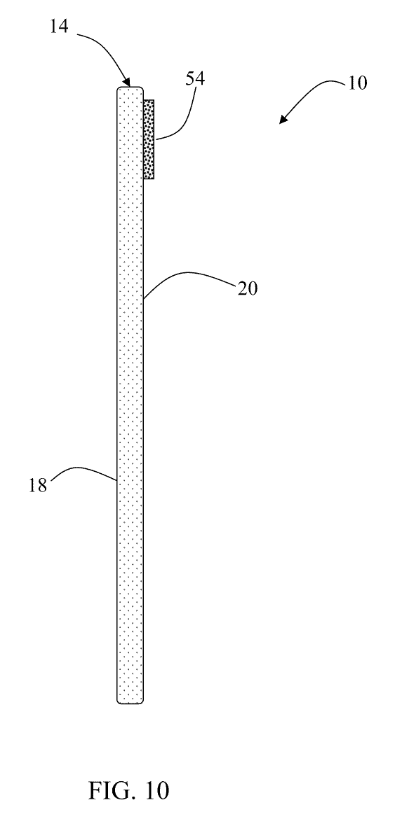

In an alternative embodiment, illustrated in FIG. 10, the fastening mechanism may comprise one or more strips of adhesive 54. Generally, the adhesive strip(s) 54 may be disposed at any location on the spring loaded band 10. As shown in FIG. 10, an adhesive strip 54 may be disposed at one end of the bottom layer 20 of the hygienic coating 14 such that the strip 54 comes into contact with and adheres to the top layer 18 of the hygienic coating 14 as the band 10 is wrapped or coiled around individual's body part. It should be appreciated that the adhesive strip 54 may include a protective cover (not illustrated), removably secured to the strip 54, which may be pulled off by an emergency responder so as to expose the adhesive prior to securing the band 10 to an individual. Additionally, the adhesive strip 54 may be configured such that, once the strip 54 has been adhered to a portion of the band 10, attempts to subsequently remove the band 10 would either damage the band (e.g. by leaving an adhesive residue or destroying the surface of the band) or make the adhesive inoperable. Such a configuration can indicate to an emergency responder or any other person using the bands that the band 10 has been tampered with or previously removed.

It should be appreciated that the present subject matter also encompasses a method for identifying and tracking individuals during patient triage, at a hospital or during a mass evacuation. The method generally includes affixing a spring loaded band 10 to an individual by applying a force F (FIG. 4) to the band 10 so as to initiate a self-transition of the band 10 from a second stable position 28 to a first stable position 26. In general, the band 10 may be configured to coil lengthwise around the individual's wrist, arm, ankle or leg while transitioning to the first stable position 26. The method also includes assigning a patient identifier number 16 associated with the band 10 to the individual so as to permit the individual to be identified and tracked.

In another embodiment of the present subject matter, the method may further include selecting the spring loaded band 10 to be affixed to an individual so that the color of the band 10 corresponds to the individual's condition or status. In such an embodiment, the color of a band 10 may be varied to correspond to a particular condition or triage status.

In a further embodiment of the present subject matter, the method may further include securing a color coded label 36 to the spring loaded band 10 so as to indicate or update an individual's condition or triage status. In such case, the color of the color coded label 36 may be varied to correspond to a particular condition or triage status.

In yet another embodiment of the present subject matter, the method may further include securing a removable tag 34 to the spring loaded band 10, wherein the patient identifier number 16 may be disposed on the removable tag 34. Additionally, the removable tag 34 may include a barcode 22 and/or a RFID tag 24 to facilitate identification and tracking

In still a further embodiment of the present subject matter, the method may further include removing tabs 40 from the detachable extension 38 of the spring loaded band 10 so as indicate or update an individual's condition or triage status. Additionally, the method may include sensing an individual's vita health information with a biometric sensor 46 disposed on the band 10. Further, the method may include securing a fastening mechanism to the band 10.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A spring loaded band for use in patient triage, hospital tracking, and mass evacuations, the band comprising:
a spring element movable between a first stable position and a second stable position, said spring element configured to coil lengthwise around an individual's wrist, arm, ankle or leg while transitioning from said second stable position to said first stable position;
a hygienic coating configured to cover said spring element; and
a unique patient identifier number disposed on said band.

2. The spring loaded band of claim 1, wherein said spring element comprises a metal or plastic bi-stable ribbon spring.

3. The spring loaded band of claim 1, wherein said hygienic coating comprises a hygienic plastic or vinyl coating.

4. The spring loaded band of claim 1, wherein said patient identifier number is printed, molded or etched onto said hygienic coating.

5. The spring loaded band of claim 1, comprising a barcode disposed on said hygienic coating, said barcode corresponding to said patient identifier number.

6. The spring loaded band of claim 1, wherein a color of said hygienic coating may be varied to correspond to an individual's condition or triage status.

7. The spring loaded band of claim 1, comprising a RFID tag disposed on or embedded within said band, said RFID tag configured to transmit a signal corresponding to said patient identifier number.

8. The spring loaded band of claim 1, comprising a removable tag secured to said hygienic coating, said patient identifier number being disposed on said removable tag.

9. The spring loaded band of claim 8, wherein said removable tag comprises a barcode corresponding to said patient identifier number.

10. The spring loaded band of claim 8, wherein said removable tag comprises a RFID tag configured to transmit a signal corresponding to said patient identifier number.

11. The spring loaded band of claim 1, comprising a color coded label removably secured to said hygienic coating, wherein a color of said color coded label may be varied to correspond to an individual's condition or triage status.

12. The spring loaded band of claim 1, comprising a detachable extension configured so as to be removable in stages to indicate or update an individual's condition or triage status.

13. The spring loaded band of claim 1, comprising a biometric sensor configured to sense and transmit health readings of an individual.

14. The spring loaded band of claim 1, comprising a fastening mechanism disposed on said band, wherein said fastening mechanism comprises a VELCRO strip and a corresponding mating surface or a strip of adhesive.

15. A method for identifying and tracking individuals during patient triage, at a hospital, or during a mass evacuation, the method comprising:
affixing a spring loaded band to an individual by applying a force to said band so as to initiate a self-transition of said band from a second stable position to a first stable position, wherein said band is configured to coil lengthwise around said individual's wrist, arm, ankle or leg while transitioning to said first stable position; and
assigning a patient identifier number associated with said band to said individual so as to permit said individual to be identified and tracked.

16. The method of claim 15, comprising selecting said band to be affixed to said individual so that a color of said band corresponds to said individual's condition or triage status, wherein the color of said band may be varied to correspond to a particular condition or triage status.

17. The method of claim 15, comprising securing a color coded label to said band so as to indicate or update said individual's condition or triage status, wherein a color of said color coded label may be varied to correspond to a particular condition or triage status.

18. The method of claim 15, comprising securing a removable tag to said band, wherein said patient identifier number is disposed on said removable tag.

19. The method of claim 18, wherein said removable tag comprises a barcode corresponding to said patient identifier number and a RFID tag configured to transmit a signal corresponding to said patient identifier number.

20. The method of claim 15, comprising removing tabs from a detachable extension of said band so as indicate or update said individual's condition or triage status.

21. The method of claim 15, comprising sensing an individual's vital health information with a biometric sensor disposed on said band.

22. The method of claim 15, comprising securing a fastening mechanism to said band.
